(19) United States
(12) Patent Application Publication
(10) Pub. No.: US 2026/0345138 A1
(43) Pub. Date: Nov. 5, 2026

(54) BIOPSY DEVICES, PLUGS FOR BIOPSY DEVICES, AND METHODS

(71) Applicant: BARD PERIPHERAL VASCULAR, INC., Franklin Lakes, NJ (US)

(72) Inventors: Chad C. VAN LIERE, Phoenix, AZ (US); Sarfaraz SHAMJI, San Antonio, TX (US)

(73) Assignee: BARD PERIPHERAL VASCULAR, INC., Franklin Lakes, NJ (US)

(21) Appl. No.: 19/486,578

(22) PCT Filed: Jun. 15, 2023
(86) PCT No.: PCT/US2023/025414

§ 371 Date: Nov. 21, 2025

(51) Int. Cl. A61B 10/02; A61B 90/00

(52) U.S. Cl. CPC A61B 10/0275; A61B 90/39; A61B 2010/0225; A61B 2090/3987

(57) ABSTRACT

A biopsy device includes a sample transport cannula, a sample collection basket, and a plug. The sample collection basket defines a basket volume. The sample collection basket is configured to receive a biopsied sample via the sample transport cannula. The plug is coupled to the sample collection basket. The plug includes a body, a marker and a septum. The body defines a plug lumen extending through a first end and a second end. The plug lumen is open to the basket volume during a sampling procedure. The marker is positioned within the plug lumen. The septum seal is positioned within the plug lumen proximal to the marker and is configured to maintain a vacuum pressure within the sample transport cannula during sample collection. During a marking procedure, the sample transport cannula is engaged to the plug such that the plug lumen is fluidically coupled to the sample transport cannula.

### TECHNICAL FIELD

The present specification generally relates to biopsy devices, plugs for biopsy devices, and methods and, more specifically, biopsy devices, plugs for biopsy devices, and methods for delivery of markers to a biopsy site.

### BACKGROUND

During biopsy sample acquisition, particularly with respect to breast biopsy, it may be desirable to place or leave a marker within a biopsy site, such that it may be determined where a biopsy had been taken. During biopsy, the biopsy device having a probe with a cutting device may be inserted into a target biopsy site, such as via an introducer cannula. To insert the marker, the biopsy device may need to be removed and another marker delivery device inserted into the resulting biopsy site. The switching of instruments between the biopsy delivery device and the marker delivery device may be cumbersome, may jostle the subject, or may otherwise be undesirable.

### SUMMARY

Embodiments of the present disclose are directed to biopsy devices, plugs for biopsy devices, and methods which allow for delivery of a marker without removal of the biopsy device from a biopsy site, thereby improving biopsy processes, procedures, and marking.

In one embodiment, a biopsy device includes a sample transport cannula, a sample collection basket, and a plug. The sample transport cannula is configured to be fluidically coupled to a vacuum source. The sample collection basket defines a basket volume in configured to be in fluidic communication with the sample transport cannula during a sampling procedure. The sample collection basket is configured to receive a biopsied sample via the sample transport cannula. The plug is configured to be coupled to the sample collection basket and is configured to seal a proximal end of the sample collection basket. The plug includes a body, a marker and a septum. The body extends between a first end and a second end and defines a plug lumen extending through the first end and the second end. The plug lumen is open to the basket volume at the second end during the sampling procedure. The marker is positioned within the plug lumen. The septum seal is positioned within the plug lumen proximal to the marker. The septum seal is configured to maintain a vacuum pressure within the sample transport cannula during sample collection, wherein during a marking procedure, the sample transport cannula is configured to be engaged to the plug such that the plug lumen is fluidically coupled to the sample transport cannula.

In another embodiment, a plug for a biopsy device includes a body, a marker, and a septum seal. The body is configured to be removably coupled to a sample collection basket of the biopsy device, such that the body seals a proximal end of the sample collection basket. The body extends between a first end and a second end and defines a plug lumen extending through the first end and the second end. The marker is positioned within the plug lumen. The septum seal is positioned within the plug lumen proximal to the marker.

In yet another embodiment, a method of sampling a biopsy site includes inserting a probe of a biopsy device to a target biopsy site, cutting a sample from the target biopsy site, transporting with vacuum pressure the sample via a sample transport cannula of the biopsy device to a sample collection basket defining a basket volume in fluidic communication with the sample transport cannula; and retracting the sample transport cannula into contact with a plug coupled to the sample collection basket. The plug includes a body extending between a first end and a second end, the body defining a plug lumen extending through the first end and the second end, wherein the sample transport cannula is in fluidic communication with the plug lumen when retracted into contact with the plug; a marker positioned within the plug lumen; and a septum seal positioned within the plug lumen proximal to the marker, the septum seal configured to maintain the vacuum pressure within the sample transport cannula during sample collection. The method further includes advancing a push rod through the septum seal into contact with the marker and pushing the marker with the push rod through the sample transport cannula to the target biopsy site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a biopsy device including a plug, a sample collection basket, and a sample transport cannula, according to one or more embodiments shown and described herein;

FIG. 2A schematically depicts a partial longitudinal cross-section of the biopsy device of FIG. 1, according to one or more embodiments shown and described herein;

FIG. 2B schematically depicts a partial longitudinal cross-section of the biopsy device of FIG. 1 at tip of the transport cannula biopsying a sample, according to one or more embodiments shown and described herein;

FIG. 3 schematically depicts the plug of FIG. 1 in isolation from the biopsy device, according to one or more embodiments shown and described herein;

FIG. 4 schematically depicts a longitudinal cross-section of the plug of FIG. 2, according to one or more embodiments shown and described herein;

FIG. 5 schematically depicts withdrawal of the sample transport cannula into contact with the plug, according to one or more embodiments shown and described herein;

FIG. 6 schematically depicts insertion of a push rod through the plug of FIG. 4 and into contact with a marker positioned within the plug, according to one or more embodiments shown and described herein;

FIG. 7 schematically depicts deployment of the marker of FIG. 6, according to one or more embodiments shown and described herein; and

FIG. 8 depicts a flowchart illustrating a method of sampling a biopsy site, according to one or more embodiments shown and described herein.

### DETAILED DESCRIPTION

The figures generally illustrate various embodiments of a biopsy device, plug, and/or method which may be used to deliver a marker directly to a biopsy site without need to remove the biopsy device from the biopsy site. For instance in embodiments, a biopsy device includes a sample transport cannula, a sample collection basket, and a plug. The plug may be mounted to the biopsy device such as during and/or after retrieval of a biopsy sample. The plug includes a body defining a plug lumen, a marker positioned within the plug lumen, and a septum seal. The marker may be advanced from the plug lumen along the sample transport cannula directly to the biopsy device. The septum seal may maintain vacuum pressure within the biopsy device allowing the plug to be coupled to the biopsy device during or after sample transportation. Accordingly, users of biopsy device as provided herein will not be required to remove the biopsy device from a biopsy site prior to insertion of the marker. Moreover, inclusion of the plug will maintain vacuum pressure, thereby allowing continued use of the biopsy device and biopsy functionality while the plug is attached. These and additional benefits will be described in greater detail below.

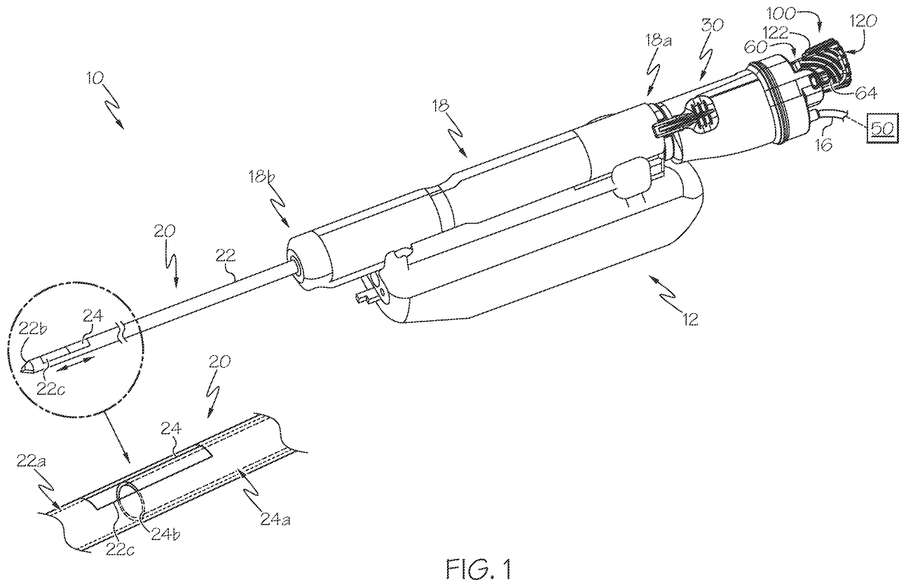

Referring now to FIG. 1, a biopsy device 10 is schematically depicted. As will be described in greater detail herein, the biopsy device 10 may generally include a sample transport cannula 20, a sample collection basket 30, and a plug 100. The biopsy device 10 may include a greater or fewer number of components without departing from the scope of the present disclosure. For example, the biopsy device 10 may further include a housing 18, a cradle 12, a vacuum line 16, etc.

The sample transport cannula 20 may collect and/or transport samples from a target location within a subject to the sample collection basket 30. In embodiments, the sample transport cannula 20 may include one or more cannulas nested within one another. For example, the sample transport cannula 20 may include an outer cannula 22 and a cutting cannula 24. However, in some embodiments, the cutting cannula 24 may be the outer cannula 22 and there may be an inner cannula within the cutting cannula 24 having features similar to the outer cannula 22 described in further detail below.

The outer cannula 22 may define an outer cannula lumen 22a and a piercing tip 22b. For example, the outer cannula 22 may be configured to pierce tissue to reach a desired biopsy site. The outer cannula 22 may have or define an opening 22c, such as radial opening 22c arranged in a sidewall of the outer cannula 22. The opening 22c may provide a sample collection window within which tissue may be drawn, such as under vacuum pressure, and cut, such as via the cutting cannula 24.

In embodiments, the cutting cannula 24 may be positioned within the outer cannula lumen 22a and movable relative to the opening 22c. For example, the cutting cannula 24 may be connected to any number of gears, drive mechanisms, motors, or the like to facilitate longitudinal a cutting motion. The cutting cannula 24 may define a cutting cannula lumen 24a extending therethrough and a distal cutting edge 24b (e.g., a sharp edge) configured to engage and cut through tissue extending into the opening 22c.

The sample transport cannula 20, such as the outer cannula 22 and/or the cutting cannula 24, maybe formed of any number of suitable materials such as stainless steel, brass nickel, aluminum, etc.

The sample transport cannula 20 may be mounted to the housing 18, which may house or hold various components of the biopsy device 10 relative to one another and/or support motion of one or more components. For example, the biopsy device 10 may house any number of motion devices (e.g., motors, actuators, drives, gears, bearings, etc.) to facilitate longitudinal and/or rotational movement of the sample transport cannula 20, such as the outer cannula 22 and/or the cutting cannula 24. For example, a first set of motion devices (e.g. a motor and one or more actuators) may be mechanically connected to the sample transport cannula 20, such as the outer cannula 22 and/or the cutting cannula 24, such that movement of the first set motion devices (e.g. movement of the actuator(s) driven by the motor) causes longitudinal motion of the sample transport cannula 20 (e.g. relative longitudinal motion of the cutting cannula 24 together with, or relative to, the outer cannula 22). Similarly, a second set of motion devices (e.g. a motor and one or more actuators) may be mechanically connected to the sample transport cannula 20, such as the outer cannula 22 and/or the cutting cannula 24, such that movement of the second set motion devices (e.g. movement of the actuator(s) driven by the motor) causes rotational motion of the sample transport cannula 20 (e.g. rotational motion of the cutting cannula 24 together with, or relative to, the outer cannula). The housing 18 may further include one or more controllers and user input devices (e.g., buttons, switches, toggles, etc.), not shown, which communicate with the motion device(s) to actuate movement of the cutting cannula 24, such as to perform a cutting operation, and/or the outer cannula 22 such as to perform a piercing operation (e.g., urge the outer cannula 22 forward to pierce tissue) or a rotation operation (such as to rotate a position of the opening 22c). As will be described in greater detail herein, movement of the sample transport cannula 20 may also be controlled, such as via a user input or automatically, in response to performing a cutting operation, to retract the sample transport cannula 20 into engagement with the plug 100 to perform a marking procedure.

The housing 18 may extend between a proximal end 18a and a distal end 18b. The sample transport cannula 20 may extend out of the distal end 18b as depicted. The housing 18 may be formed of any suitable material such as metal, plastic, or the like. In some embodiments, the housing 18 may be removably coupled to a cradle 12. In embodiments, the cradle 12 may hold capital equipment, motors, batteries, gearing, etc. which may mate with corresponding portions within the housing 18 to drive operation (e.g., movement of the sample transport cannula 20, such as the cutting cannula 24 and/or the outer cannula 22 as described above). In such embodiments, the housing 18 and components attached thereto may be disposable. In other embodiments, there may not be a cradle 12 and all components may be housed within the housing 18.

In embodiments, the cradle 12 and/or the housing 18 may house a controller (not depicted) for controlling various functionality of the biopsy device 10. In some embodiments, the controller may be remote, such as part of a console. For example, the controller may include any number of processors, shipsets, memories, or the like. The memory(ies) may store non-transitory computer-readable media providing logic for operation of the various functions of the biopsy device 10. For example, piercing, cutting, and/or marker delivery logic. The controller may be communicatively coupled to the motion devices and the user input devices, such as via wired or wireless communication. Each piece of logic may cause the sample transport cannula 20 to move with the motion device(s). For example, when executing piercing logic, the motion devices may be controlled by the controller to accelerate the outer cannula 22 forward to pierce target tissue; when executing cutting logic, the controller may cause the cutting cannula 24 to move forward to slice through tissue protruding through the opening 22c; when executing marking logic, the controller may cause the sample transport cannula 20 (e.g., the cutting cannula 24) to retract into contact with the plug 100 as will be described in greater detail below. As will be described in greater detail below, in embodiments, the controller may be communicatively coupled to a vacuum source 50. The controller may operate the vacuum source 50, such as during cutting logic execution to first draw tissue into the opening 22c, cut the tissue with the cutting cannula 24, and pull, with vacuum pressure, the sample to the awaiting sample collection basket 30.

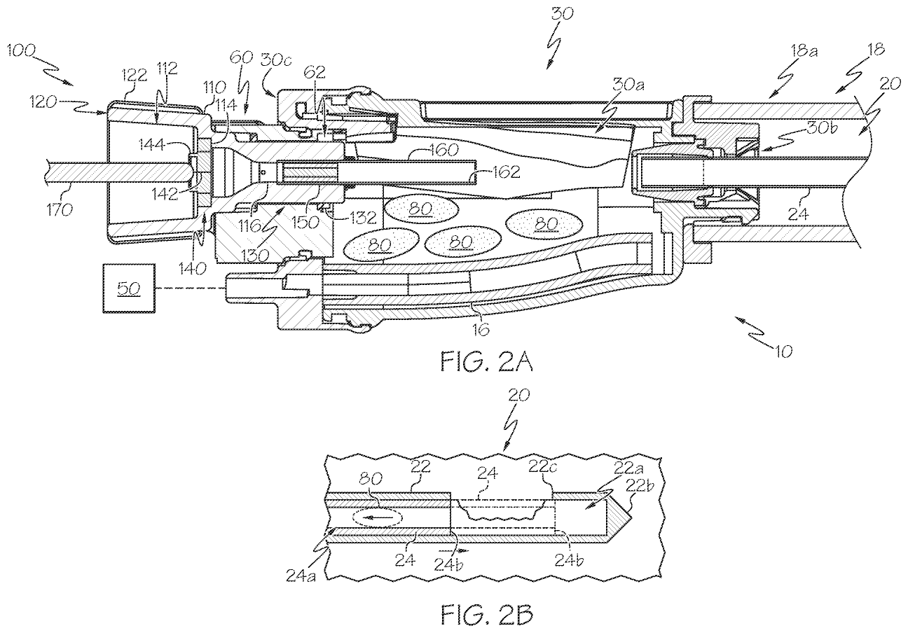

Referring to FIG. 2A, a longitudinal cross-section of the biopsy device 10 including the sample collection basket 30 is depicted. As illustrated, in embodiments, the sample collection basket 30 may be positioned to receive a proximal end of the sample transport cannula 20, such as a proximal end of the cutting cannula 24, so as to be able to receive tissue samples from the sample transport cannula 20 (e.g., the cutting cannula 24) during sample acquisition. The sample collection basket 30 defines a basket volume 30a which, as illustrated, is in fluidic communication with the sample transport cannula 20 (e.g., the cutting cannula 24, or the lumen 24a) during a sampling procedure (such as during execution of cutting logic described above). For example, a proximal end of the cutting cannula 24 may be positioned within or adjacent the sample collection basket 30 such that samples are deposited directly within the sample collection basket 30.

As depicted, the sample collection basket 30 may be mounted to a proximal end 18a of the housing 18. For example, the sample collection basket 30 may be removably coupled to the housing 18 via any suitable detachable coupling mechanism such that the sample collection basket 30 may be removed, such as to empty of samples 80, and replaced as desired. The sample collection basket 30 may be formed of any number of suitable materials (e.g., metal, plastic, ceramic, etc.). The detachable coupling mechanism may include, for example, engageable male and female screw threads on the sample collection basket 30 and the housing 18, or the like.

The sample collection basket 30 may have a distal opening 30b and a proximal opening 30c. The distal opening 30b may be sized to receive the proximal end of the sample transport cannula 20 (e.g., the cutting cannula 24). In such embodiments, the sample collection basket 30 may seal to a proximal end 18a of the housing 18 about the opening 22c, which may allow vacuum to be applied therethrough to the sample transport cannula 20 when the biopsy device 10 is assembled, and prevent vacuum leakage between the housing 18 and the sample collection basket 30. For example, the vacuum line 16 may be coupled to the sample collection basket 30 and/or extend within the sample collection basket 30 so as to be in fluidic communication with the basket volume 30a. The vacuum line 16 may be coupled to a vacuum source 50 (e.g., pump) to pull vacuum through the sample transport cannula 20. Accordingly, vacuum may be used to move tissue along the sample transport cannula 20 and into the sample collection basket 30.

The proximal opening 30c may be selectively sealed such as with the plug 100. That is, the plug 100 may be configured to fluidically seal against edges of the opening 30c so as to provide a fluid-tight seal therebetween. Accordingly, vacuum provided through the sample collection basket 30 and the sample transport cannula 20 may be maintained to pull material proximally through the sample transport cannula 20.

In some embodiments, coupled to or included as part of the sample collection basket 30 may be a mount 60 that may couple the plug 100 to the sample collection basket 30. For example, the mount 60 may be received within the proximal opening 30c and the mount 60 may itself define a through-hole 62 which receives the plug 100. In such embodiments, the mount 60 may seal to the sample collection basket 30, such as to the edges of the proximal opening 30c, by any traditional mechanisms such as gaskets, O-rings, or the like. The mount 60 may be made from the same or different materials from the sample collection basket 30. The mount may be removable from the sample collection basket 30 or permanently affixed thereto.

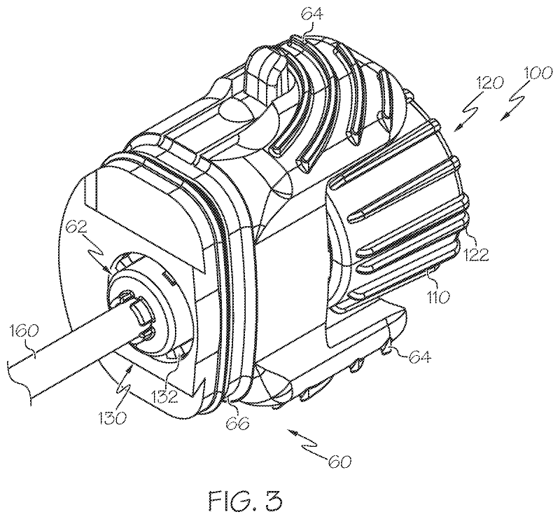

Referring to FIG. 3, the plug 100 and mount 60 are depicted in isolation. The mount 60 may have one or more engagement wings 64 or interfaces which may be engaged by a user to assist in coupling or removal of the mount 60 from the sample collection basket 30. The one or more engagement wings 64 may include a first engagement wing and a second engagement wing which extend proximally from a main body portion 66 of the mount 60 for example. The first engagement wing and the second engagement wing may be arranged in radial opposition to one another as depicted, though other arrangements are contemplated and possible. The first and second engagement wings may include surface texture/features (e.g., ribs, grooves, patterns, etc.), which assist a use in grasping and manipulating the mount 60.

The plug 100 may be received within the mount 60, such as between the one or more engagement wings 64 as shown. The plug 100 may seal to the through-hole 62. For example, the plug 100 may be received within the through-hole 62 and seal against a wall of the mount 60 defining the through-hole 62. The plug 100 may be formed of any material, such as rubber, plastic or the like. The plug 100 may be formed of a material which directly forms a seal with the mount 60 or an O-ring, gasket, or similar material may be positioned between the plug 100 and the mount 60. Accordingly, the plug 100 provides a seal (e.g., a fluid-tight seal) to the proximal end 18a of the sample collection basket 30, for example, through further interaction with the mount 60 as noted above.

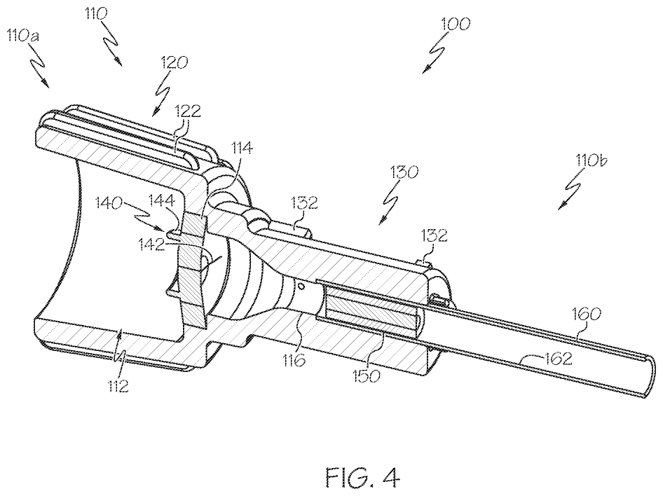

Referring to FIG. 4, a longitudinal cross-section of the plug 100 is generally depicted in isolation. The plug 100 generally includes a body 110 extending between a first end 110a and a second end 110b. The body 110 defines a plug lumen 112 extending through the first end 110a and the second end 110b. The second end 110b of the plug 100, when assembled to the sample collection basket 30, may be positioned adjacent or within the basket volume 30a, such as illustrated in FIG. 2. Accordingly, the plug lumen 112 is open to the basket volume 30a throughout a sampling procedure. That is, the plug lumen 112 as provided at the second end 110b of the plug 100 is illustrated as open to the basket volume 30a during sampling.

Referring again to FIG. 4, in embodiments, the body 110 may define a user engagement portion 120. Extending from the user engagement portion 120 may be an insertion portion 130, which gets inserted into the mount 60 and/or the sample collection basket 30, as illustrated in FIG. 2. Still referring to FIG. 4, the user engagement portion 120 may have a larger outer diameter or outer profile than the insertion portion 130. For example, the user engagement portion 120 may include one or more, such as a plurality of surface features 122 (e.g., ribs, grooves, patterns, etc.) extending from a surface thereof, which may assist in gripping and manipulation by a user.

In embodiments, the plug 100 may couple to the sample collection basket 30 and/or the mount 60 via a quarter turn arrangement, a threaded arrangement, a press-fit, or the like. In embodiments, the insertion portion 130 may include one or more keys 132 that may extend from a surface of the insertion portion 130 and engage a mating channel (not shown) formed within the mount 60 of the sample collection basket 30, such as in a quarter turn arrangement.

A septum seal 140 is positioned within the plug lumen 112. For example, the septum seal 140 may extend laterally across the plug lumen 112, providing a fluid seal against unwanted fluid leakage through the plug lumen 112. That is, the septum seal 140 maintains a vacuum pressure within the sample transport cannula 20 during sample collection. The septum seal 140 may be spaced from both the first end 110a and the second end 110b of the body 110 as shown.

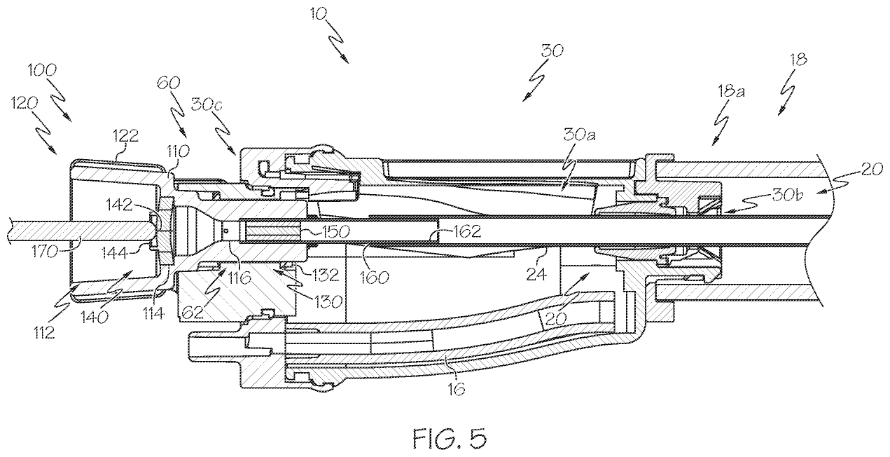

In embodiments, the septum seal 140 may have a pre-cut opening or pre-made slit 142 formed therein, such as may receive a push rod. The slit 142 may extend laterally across the septum. The slit 142 may be any size, but the edges of the slit 142 may mate, meet, or overlap, to maintain the seal, until broken or opened, as will be described in great detail herein. In embodiments, to assist with maintaining the seal, the septum seal 140 may be convexly curved toward a proximal end (e.g., the first end 110a) of the plug 100, under the absence of vacuum (shown in FIG. 5). Accordingly, when vacuum is applied (depicted in FIG. 2), the septum seal 140 may be pulled, somewhat distally, which may flatten the septum seal 140, thereby providing more force to seal the slit 142 against leakage. In some embodiments, the slit 142, may not extend fully through the septum seal 140, but may instead be a perforation, or scoring providing a point of weakness which may be broken via such as insertion of a push rod therethrough.

In embodiments, the septum seal 140 may include one or more ridges 144 extending in a proximal direction. For example, the one or more ridges 144 may be positioned adjacent the slit 142 and may provide increased structure for maintaining the fluidic seal through the plug 100, for example for better withstanding vacuum pressure exerted in a distal direction. In embodiments, the one or more ridges 144 may be a circumferences ridge surrounding or partially surrounding the slit 142. In embodiments, the one or more ridges 144 may provide a guide for guiding a push rod through the slit 142.

The septum seal 140 may be formed of any suitable material, rubber, silicone, or the like. The septum seal 140 may be sized to fill the lateral cross-sectional area of the plug 100 where it is positioned. Accordingly, the septum seal 140 may have any suitable shape to seal off the lateral cross-section of the plug lumen 112 where it is positioned.

Still referring to FIG. 4, a marker 150 is positioned within the plug lumen 112. For example, the marker 150 may be pre-positioned within the plug lumen 112 distal to the septum seal 140, as depicted. That is the marker 150 may be in position throughout a biopsy procedure. The marker 150 may be any sort of marker 150 suitable for marking a biopsy site. For example, the marker 150 may be a metal marker, plastic marker, or the like having any suitable marker shape.

The plug lumen 112 may have regions of multiple sizes or diameters. For example, a portion of the plug lumen 112 positioned proximal to the septum seal 140 may be larger than a diameter of the plug lumen 112 at a position distal to the septum seal 140. In such embodiments, a shelf 114 may be provided via the changing diameter. The shelf 114 may provide a location to anchor or attach the septum seal 140, such as via adhesive, fasteners, or the like. In various embodiments, one or more portions of the plug lumen 112 may be tapered, such as distally tapered, thereby providing a tapering cross-section. Such tapering cross-section may provide guidance for a push rod 170 toward the marker 150 positioned within the plug lumen 112 as shown. That is a portion of the plug lumen 112, such as distal to the septum seal 140, may taper toward a portion of the plug lumen 112 in which the marker 150 is positioned.

Referring now to FIG. 5, during a marking procedure, the sample transport cannula 20, such as the cutting cannula 24 is engaged to the plug 100 such that the plug lumen 112 is fluidically coupled to the sample transport cannula 20. For example, the sample transport cannula 20 (e.g., the cutting cannula 24) may be retracted (such as via the controller or manually) into contact with the plug 100 to fluidically couple the sample transport cannula 20 to the plug lumen 112, thereby providing a pathway for the marker 150 to travel from the plug lumen 112 into the sample transport cannula 20. In some embodiments, to assist in mating the sample transport cannula 20 to the plug lumen 112, the plug lumen 112 may include a mating cannula 160 positioned within the plug lumen 112 and configured to be received by the sample transport cannula 20 (e.g., the cutting cannula 24) during the marking procedure. For example, the mating cannula 160 may be a rigid cannula that acts to extend the plug lumen 112. In other embodiments, the mating cannula 160 may be flexible. The mating cannula 160 may be formed of any material such as but not limited to, stainless steel, plastic, other metal, or the like. As illustrated the mating cannula 160 may be recessed into the body 110 of the plug 100, such as within the plug lumen 112. In such embodiments, a transition between an inner surface 116 of the plug lumen 112 and an inner surface 162 of the mating cannula 160 may be substantially smooth or not stepped therebetween, which may assist in facilitating guidance of the push rod through the plug lumen 112 and the mating cannula 160. In some embodiments, the marker 150 is prepositioned within the mating cannula 160, such as within a portion mounted within the plug lumen 112.

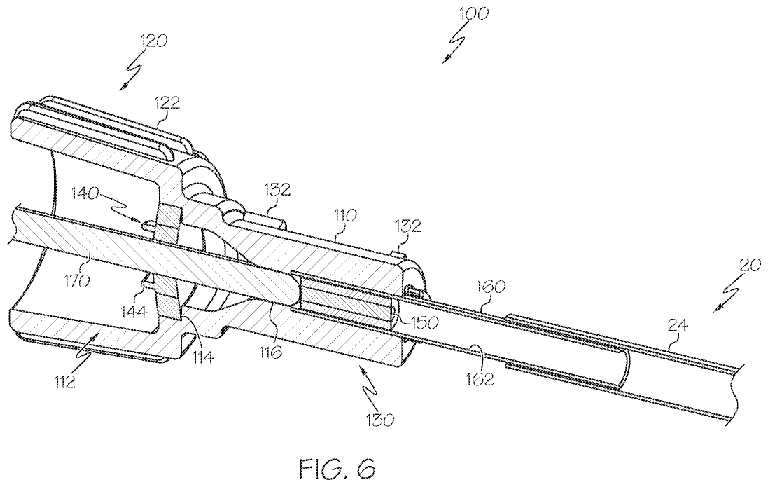

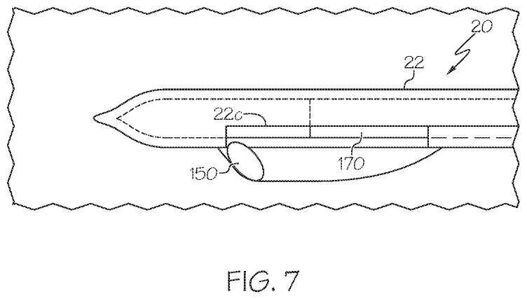

In embodiments, the biopsy device 10 further includes the push rod 170, such as provided within the biopsy device 10 as a kit. The push rod 170 may be sized to be advanced through the septum seal 140 and push the marker 150 during the marking procedure from the plug lumen 112 through the sample transport cannula 20 (such as through the cutting cannula 24 and/or the outer cannula 22) and out of the biopsy device 10, such as illustrated in FIG. 6. For example, as the push rod 170 is advanced through the septum seal 140, the push rod 170 may pierce the septum seal 140 and be pushed further into contact with the marker 150. Once in contact with the marker 150, the push rod 170 may be further advanced to advance the marker 150 distally through the sample transport cannula 20 (such as the cutting cannula 24, and out of the window of the outer cannula 22 for example and as illustrated in FIG. 7.

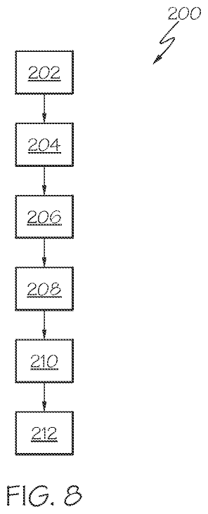

FIG. 8 depicts a flow chart illustrating a method 200 of using a biopsy device 10 including a plug 100 a described herein. Such method 200 may include a greater or fewer number of steps, and the various steps may be performed in various orders and not necessarily the order depicted. At step 202, a probe (e.g., the sample transport cannula 20, the outer cannula 22, and/or the cutter cannula) may be inserted to a target biopsy site (e.g., such as within a breast of a subject). Such insertion is generally depicted in FIG. 2. For example, the sample transport cannula 20 such as the outer cannula 22, may be used to pierce the target tissue and be embedded into the subject at a desired location. Visual guidance may happen under ultrasound, x-ray, or the like. As noted above, insertion may be accomplished via execution of piercing logic by a controller to propel to outer cannula 22 into target tissue. A user may cause execution of piercing logic via operation of a user input device.

At step 204 the method 200 may include cutting a sample from the target biopsy site. As noted above, cutting logic may be executed by the controller (such as in response to operation of a user input device), to actuate the cutting cannula 24. For example, the sample window may be opened, such as via retraction of the cutting cannula 24. Vacuum may be activated, such as automatically by the controller or manually, to draw in tissue through the opening 22c. The cutting cannula 24 may then be advanced distally to cut a tissue sample 80 pulled within the opening 22c. Under step 206, the sample 80 may then be transported, such as under vacuum, proximally to the sample collection basket 30, wherein the sample 80 drops into the sample collection basket 30. In embodiments, multiple samples 80 may be taken from a location and provided within the sample collection basket, as illustrated in FIG. 2.

At step 208, the sample transport cannula 20 may be withdrawn further into contact with the plug 100 in preparation for marking. For example, the controller may automatically execute marking logic in response to executing cutting logic. Accordingly, the controller may cause retraction of the sample transport cannula 20 into contact with the plug 100. In some embodiments, retraction may involve withdrawing the sample transport cannula 20 such that the mating cannula 160 is positioned within the sample transport cannula 20, such as within the cutting cannula 24, such as illustrated in FIG. 4. In some embodiments, such steps may be manually performed.

At step 210, the method 200 may include advancing the push rod 170 through the septum seal 140 of the plug 100 into contact with the marker 150, such as illustrated in FIG. 5. At step 212 the method 200 further includes pushing the marker 150 with the push rod 170 through the sample transport cannula 20, such as the cutting cannula 24 and the outer cannula 22, to the target biopsy site, thereby marking the location of biopsy. For example, the push rod may push the marker 150 through the cutting cannula 24 and the outer cannula 22, and through the window as illustrated in FIG. 8.

In embodiments, the method 200 may further include removing the plug 100 and push rod from the biopsy device 10 and coupling a second plug 100 to the biopsy device 10. The second plug 100 may be identical to the first plug 100 and may contain a second marker 150 for subsequent delivery.

Embodiments may be further described with respect to the following numbered clauses:

1. A biopsy device comprising: a sample transport cannula configured to be fluidically coupled to a vacuum source; a sample collection basket defining a basket volume configured to be in fluidic communication with the sample transport cannula during a sampling procedure, the sample collection basket configured to receive a biopsied sample via the sample transport cannula; and a plug configured to be coupled to the sample collection basket, the plug configured to seal a proximal end of the sample collection basket, the plug comprising: a body extending between a first end and a second end, the body defining a plug lumen extending through the first end and the second end, the plug lumen open to the basket volume at the second end during the sampling procedure; a marker positioned within the plug lumen; and a septum seal positioned within the plug lumen proximal to the marker, the septum seal configured to maintain a vacuum pressure within the sample transport cannula during sample collection, wherein during a marking procedure, the sample transport cannula is configured to be engaged to the plug such that the plug lumen is fluidically coupled to the sample transport cannula.

2. The biopsy device of clause 1, further comprising a push rod configured to be advanced through the septum seal and push the marker during the marking procedure from the plug lumen through the sample transport cannula and out of the biopsy device.

3. The biopsy device of any preceding clause, wherein the septum seal comprises a pre-cut slit configured to receive a push rod.

4. The biopsy device of any preceding clause, wherein the plug comprises a mating cannula positioned within the plug lumen and configured to be received by the sample transport cannula during the marking procedure.

5. The biopsy device of clause 4, wherein the mating cannula is recessed into the body of the plug.

6. The biopsy device any preceding clause, wherein the plug lumen comprises a tapering cross-section between the first end and the second end of the body.

7. The biopsy device of any preceding clause, wherein the plug is removably coupled to the sample collection basket.

8. A plug for a biopsy device comprising: a body configured to be removably coupled to a sample collection basket of the biopsy device, such that the body seals a proximal end of the sample collection basket, the body extending between a first end and a second end, the body defining a plug lumen extending through the first end and the second end; a marker positioned within the plug lumen; and a septum seal positioned within the plug lumen proximal to the marker.

9. The plug of clause 8, wherein the septum seal comprises a pre-cut slit configured to receive a push rod.

10. The plug of any preceding clause, wherein the plug comprises a mating cannula positioned within the plug lumen and configured to be received by a sample transport cannula during a marking procedure.

11. The plug of clause 10, wherein the mating cannula is recessed into the body of the plug.

12. The plug of any preceding clause, wherein the body defines a user engagement portion comprising a plurality of surface features.

13. The plug of any preceding clause, wherein the septum seal comprises one or more ridges extending in a proximal direction.

14. The plug of any preceding clause, wherein the septum seal is position between and spaced from both the first end and the second end of the body.

15. A method of sampling a biopsy site, the method comprising: inserting a probe of a biopsy device to a target biopsy site; cutting a sample from the target biopsy site; transporting with vacuum pressure the sample via a sample transport cannula of the biopsy device to a sample collection basket defining a basket volume in fluidic communication with the sample transport cannula; and retracting the sample transport cannula into contact with a plug coupled to the sample collection basket, wherein the plug comprises: a body extending between a first end and a second end, the body defining a plug lumen extending through the first end and the second end, wherein the sample transport cannula is in fluidic communication with the plug lumen when retracted into contact with the plug; a marker positioned within the plug lumen; and a septum seal positioned within the plug lumen proximal to the marker, the septum seal configured to maintain the vacuum pressure within the sample transport cannula during sample collection; and advancing a push rod through the septum seal into contact with the marker; and pushing the marker with the push rod through the sample transport cannula to the target biopsy site.

16. The method of clause 15, further comprising: removing the plug and the push rod from the biopsy device; and coupling a second plug to the biopsy device, the second plug comprising a second marker for delivery.

17. The method of any preceding clause, wherein advancing the push rod through the septum seal comprises pushing the push rod through a pre-made slit within the septum seal.

18. The method of any preceding clause, wherein the marker is positioned within a mating cannula positioned within the plug lumen, and retracting the sample transport cannula into contact with the plug comprises receiving the mating cannula within the sample transport cannula.

19. The method of any preceding clause, wherein the sample transport cannula is a cutting cannula.

20. The method of any preceding clause, wherein the septum seal comprises one or more ridges extending in a proximal direction.

It should now be understood that embodiments of the present disclosure are directed to biopsy devices, plugs, and/or methods which may be used to deliver a marker directly to a biopsy site without need to remove the biopsy device from the biopsy site. For instance embodiments include a plug with a pre-mounted marker. The plug provides a seal such that vacuum pressure may be maintained throughout a biopsy procedure. Accordingly, a separate marker device is not needed, which simplifies biopsy and marking procedures.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A biopsy device comprising:
a sample transport cannula configured to be fluidically coupled to a vacuum source;
a sample collection basket defining a basket volume configured to be in fluidic communication with the sample transport cannula during a sampling procedure, the sample collection basket configured to receive a biopsied sample via the sample transport cannula; and
a plug configured to be coupled to the sample collection basket, the plug configured to seal a proximal end of the sample collection basket, the plug comprising:
a body extending between a first end and a second end, the body defining a plug lumen extending through the first end and the second end, the plug lumen open to the basket volume at the second end during the sampling procedure;
a marker positioned within the plug lumen; and
a septum seal positioned within the plug lumen proximal to the marker, the septum seal configured to maintain a vacuum pressure within the sample transport cannula during sample collection, wherein during a marking procedure, the sample transport cannula is configured to be engaged to the plug such that the plug lumen is fluidically coupled to the sample transport cannula.

2. The biopsy device of claim 1, further comprising a push rod configured to be advanced through the septum seal and push the marker during the marking procedure from the plug lumen through the sample transport cannula and out of the biopsy device.

3. The biopsy device of claim 1, wherein the septum seal comprises a pre-cut slit configured to receive a push rod.

4. The biopsy device of claim 1, wherein the plug comprises a mating cannula positioned within the plug lumen and configured to be received by the sample transport cannula during the marking procedure.

5. The biopsy device of claim 4, wherein the mating cannula is recessed into the body of the plug.

6. The biopsy device claim 1, wherein the plug lumen comprises a tapering cross-section between the first end and the second end of the body.

7. The biopsy device of claim 1, wherein the plug is removably coupled to the sample collection basket.

8. A plug for a biopsy device comprising:
a body configured to be removably coupled to a sample collection basket of the biopsy device, such that the body seals a proximal end of the sample collection basket, the body extending between a first end and a second end, the body defining a plug lumen extending through the first end and the second end;
a marker positioned within the plug lumen; and
a septum seal positioned within the plug lumen proximal to the marker.

9. The plug of claim 8, wherein the septum seal comprises a pre-cut slit configured to receive a push rod.

10. The plug of claim 8, wherein the plug comprises a mating cannula positioned within the plug lumen and configured to be received by a sample transport cannula during a marking procedure.

11. The plug of claim 10, wherein the mating cannula is recessed into the body of the plug.

12. The plug of claim 8, wherein the body defines a user engagement portion comprising a plurality of surface features.

13. The plug of claim 8, wherein the septum seal comprises one or more ridges extending in a proximal direction.

14. The plug of claim 8, wherein the septum seal is position between and spaced from both the first end and the second end of the body.

15. A method of sampling a biopsy site, the method comprising:
inserting a probe of a biopsy device to a target biopsy site;
cutting a sample from the target biopsy site;
transporting with vacuum pressure the sample via a sample transport cannula of the biopsy device to a sample collection basket defining a basket volume in fluidic communication with the sample transport cannula; and
retracting the sample transport cannula into contact with a plug coupled to the sample collection basket, wherein the plug comprises:
a body extending between a first end and a second end, the body defining a plug lumen extending through the first end and the second end, wherein the sample transport cannula is in fluidic communication with the plug lumen when retracted into contact with the plug;
a marker positioned within the plug lumen; and
a septum seal positioned within the plug lumen proximal to the marker, the septum seal configured to maintain the vacuum pressure within the sample transport cannula during sample collection; and
advancing a push rod through the septum seal into contact with the marker; and
pushing the marker with the push rod through the sample transport cannula to the target biopsy site.

16. The method of claim 15, further comprising:
removing the plug and the push rod from the biopsy device; and
coupling a second plug to the biopsy device, the second plug comprising a second marker for delivery.

17. The method of claim 15, wherein advancing the push rod through the septum seal comprises pushing the push rod through a pre-made slit within the septum seal.

18. The method of claim 15, wherein the marker is positioned within a mating cannula positioned within the plug lumen, and retracting the sample transport cannula into contact with the plug comprises receiving the mating cannula within the sample transport cannula.

19. The method of claim 15, wherein the sample transport cannula is a cutting cannula.

20. The method of claim 15, wherein the septum seal comprises one or more ridges extending in a proximal direction.
